(19) United States
(12) Patent Application Publication
(10) Pub. No.: US 2025/0127646 A1
(43) Pub. Date: Apr. 24, 2025

(54) LINGERIE

(71) Applicant: Olena SYMONOVA, Miami Beach, FL (US)

(72) Inventor: Olena SYMONOVA, Miami Beach, FL (US)

(21) Appl. No.: 18/491,011

(22) Filed: Oct. 20, 2023

(51) Int. Cl. A61F 5/37; A41B 9/00

(52) U.S. Cl. CPC A61F 5/3761; A41B 9/00

(57) ABSTRACT

A piece of lingerie. The piece of lingerie can include a main body of the piece of lingerie, and at least one binding strap fixedly or removably coupled to the main body. When the lingerie is worn on a body of a wearer, the binding strap can include a tension anchor proximate to the body of the wearer and at least one free binding end extending distally from the tension anchor. The binding end can be connected to the main body of the piece of lingerie at or via the tension anchor. The tension anchor can be disposed at or proximate a specific anatomical location on the body of the wearer, and the binding end of the binding strap can have a length sufficient to bind at least one limb of the wearer to said limb or to another limb of the wearer.

### BACKGROUND

The embodiments disclosed herein are directed to lingerie, specifically, lingerie that may be used in an intimate setting, and, more specifically, wherein individuals may desire to include bondage elements in the intimate setting.

Various known implements have been provided for including bondage in intimate settings, with the most well-known of these being ropes, belt-type straps, cuffs, and so forth. Such implements all provide differing sensations for the user, as well as different possible positions and so forth. A disadvantage of many known implements is that they may provide a feeling of discomfort. For example, rope, such as used in shibari-style bondage, may pinch nerves and blood vessels, and impart significant pressure to muscles, both due to the style of bondage and due to the high pressure of the narrow rope that impinges on the body.

Some intimate partners may not desire such effects, and may rather desire a more relaxed form of bondage that allows an increase in relaxation and intimacy. Furthermore, an aesthetic effect may be enhanced by including attractive lingerie in the intimate setting. Additionally, by combining the bondage elements and the lingerie, bodily sensations, physiological and psychological effects, and intimacy may further be enhanced.

Accordingly, a piece of lingerie including bondage elements for use in an intimate is desired.

### SUMMARY

According to at least one exemplary embodiment, a piece of lingerie is disclosed. The piece of lingerie can include a main body of the piece of lingerie, and at least one binding strap fixedly or removably coupled to the main body. When the lingerie is worn on a body of a wearer, the binding strap can include a tension anchor proximate to the body of the wearer and at least one free binding end extending distally from the tension anchor. The binding end can be connected to the main body of the piece of lingerie at or via the tension anchor. The tension anchor can be disposed at or proximate a specific anatomical location on the body of the wearer, and the binding end of the binding strap can have a length sufficient to bind at least one limb of the wearer to said limb or to another limb of the wearer.

According to another exemplary embodiment, a piece of lingerie is disclosed. The piece of lingerie can include a main body of the piece of lingerie and at least one binding strap coupled or couplable to the main body. When the lingerie is worn on a body of a wearer, the binding strap can include a tension anchor disposed at or proximate a specific anatomical location on the body of the wearer, and at least one free binding end extending distally from the tension anchor. The tension anchor can be coupled to the main body of the piece of lingerie at an attachment point or via at least one coupling element, the binding end can be connected to the piece of lingerie at or via the tension anchor, and the binding end of the binding strap can have a length sufficient to bind at least one limb of the wearer to itself or to another limb of the wearer. The specific anatomical location can be one or more of along the spine of the wearer, along or below the sternum of the wearer and collinear therewith, and along the sides of the torso of the wearer.

According to another exemplary embodiment, a method for utilizing a piece of lingerie in an intimate bondage setting is disclosed. The method can include providing a piece of lingerie having a main body and at least one binding strap coupled or couplable to the main body, donning the piece of lingerie such that a tension anchor of the binding strap is disposed at or proximate a specific anatomical location on the body of the wearer, extending at least one binding end of the binding strap distally from the tension anchor, and binding, using the binding end of the binding strap, at least one limb of the wearer to itself or to another limb of the wearer. The binding end of the binding strap can have a length sufficient to perform the binding step. The specific anatomical location can be one or more of along the spine of the wearer, along or below the sternum of the wearer and collinear therewith, and along the sides of the torso of the wearer.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows an exemplary embodiment of lingerie according to the present disclosure.

FIG. 2 shows another exemplary embodiment of lingerie according to the present disclosure.

FIG. 3A shows a rear view of an exemplary embodiment of a brassiere with a fixedly coupled binding strap.

FIG. 3B shows a rear view of an exemplary embodiment of a brassiere with a binding strap coupled thereto via a single coupling element.

FIG. 3C shows a front view of an exemplary embodiment of a brassiere with a fixedly coupled binding strap.

FIG. 3D shows a front view of an exemplary embodiment of a brassiere with a binding strap coupled thereto via a single coupling element.

FIG. 4A shows a rear view of an exemplary embodiment of a corset with a fixedly coupled binding strap.

FIG. 4B shows a rear view of an exemplary embodiment of a corset with a binding strap coupled thereto via a single coupling element.

FIG. 4C shows a front view of an exemplary embodiment of a corset with a fixedly coupled binding strap.

FIG. 4D shows a front view of an exemplary embodiment of a corset with a binding strap coupled thereto via a single coupling element.

FIG. 4E shows a left view of an exemplary embodiment of a corset with a fixedly coupled binding strap.

FIG. 4F shows a right view of an exemplary embodiment of a corset with a fixedly coupled binding strap.

FIG. 4G shows a left view of an exemplary embodiment of a corset with a binding strap coupled thereto via a single coupling element.

FIG. 4H shows a right view of an exemplary embodiment of a corset with a binding strap coupled thereto via a single coupling element.

FIG. 5A shows a rear view of an exemplary embodiment of a panties with a fixedly coupled binding strap.

FIG. 5B shows a rear view of an exemplary embodiment of a panties with a binding strap coupled thereto via a single coupling element.

FIG. 5C shows a front view of an exemplary embodiment of a panties with a fixedly coupled binding strap.

FIG. 5D shows a front view of an exemplary embodiment of a panties with a binding strap coupled thereto via a single coupling element.

FIG. 5E shows a left view of an exemplary embodiment of a panties with a fixedly coupled binding strap.

FIG. 5F shows a right view of an exemplary embodiment of a panties with a fixedly coupled binding strap.

FIG. 5G shows a left view of an exemplary embodiment of a panties with a binding strap coupled thereto via a single coupling element.

FIG. 5H shows a right view of an exemplary embodiment of a panties with a binding strap coupled thereto via a single coupling element.

FIG. 6A shows a rear view of an exemplary embodiment of a brassiere with a binding strap coupled thereto via several coupling elements.

FIG. 6B shows a front view of an exemplary embodiment of a brassiere with a binding strap coupled thereto via several coupling elements.

FIG. 7A shows a rear view of an exemplary embodiment of a corset with a binding strap coupled thereto via several coupling elements.

FIG. 7B shows a front view of an exemplary embodiment of a corset with a binding strap coupled thereto via several coupling elements.

FIG. 7C shows a left view of an exemplary embodiment of a corset with a binding strap coupled thereto via several coupling elements.

FIG. 7D shows a right view of an exemplary embodiment of a corset with a binding strap coupled thereto via several coupling elements.

FIG. 8A shows a left view of an exemplary embodiment of a panties with a binding strap coupled thereto via several coupling elements.

FIG. 8B shows a right view of an exemplary embodiment of a panties with a binding strap coupled thereto via several coupling elements.

FIG. 9A shows another exemplary embodiment of lingerie according to the present disclosure, in use.

FIG. 9B shows another exemplary embodiment of lingerie according to the present disclosure, in use.

FIG. 9C shows another exemplary embodiment of lingerie according to the present disclosure, in use.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

The embodiments disclosed herein are directed to lingerie, specifically, lingerie that may be used in an intimate setting, and, more specifically, wherein individuals may desire to include bondage elements in the intimate setting.

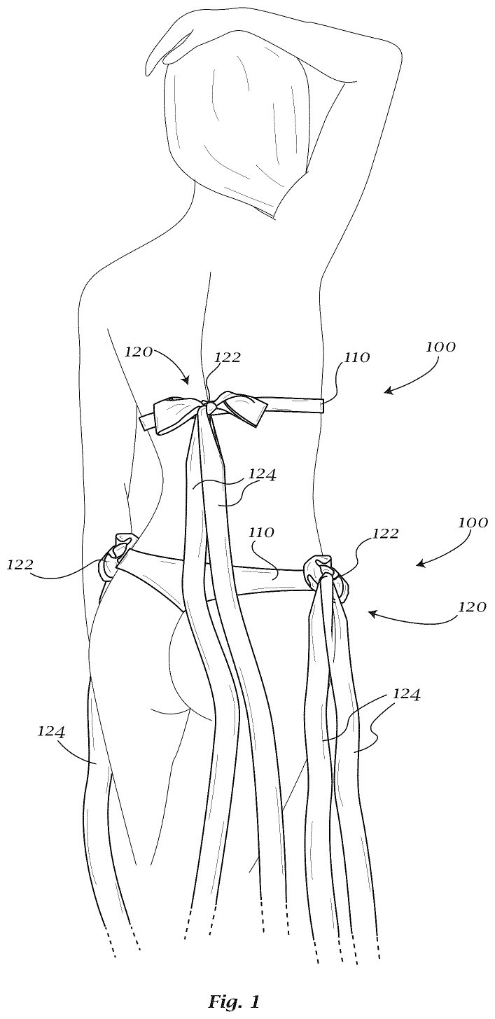

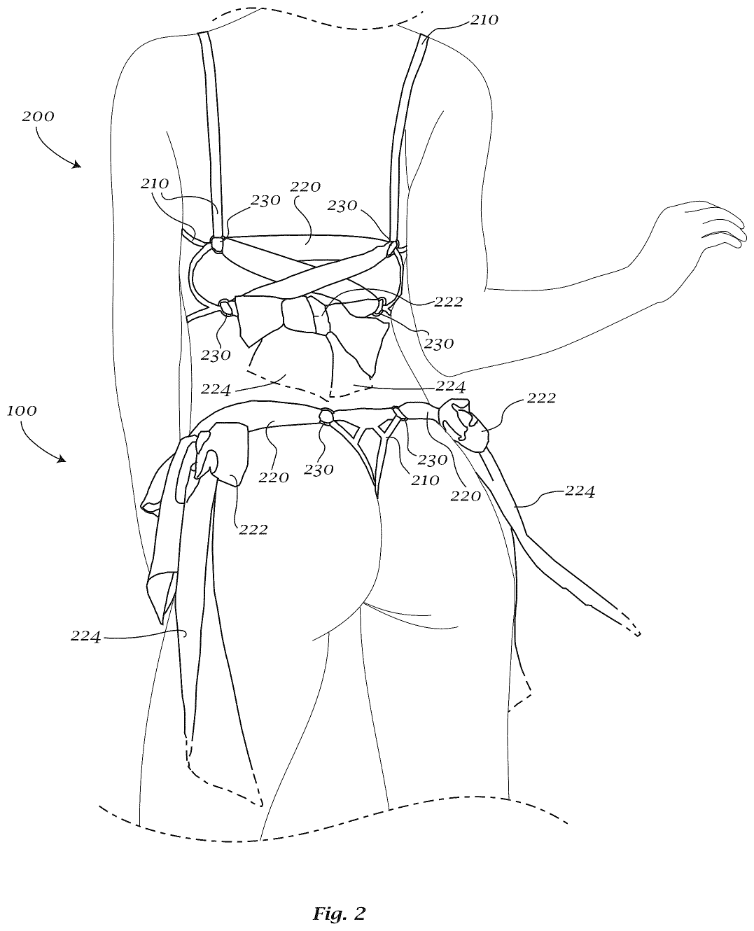

According to at least one exemplary embodiment, and referring generally to FIGS. 1-2, a piece of lingerie 100,200 that includes binding straps 120,220 coupled to the main body 110,210 of the piece of lingerie 100,200 is disclosed. When the lingerie is worn by a wearer, tension anchors 122,222 of the binding straps 120,220 may be positioned proximate specific anatomical locations on the human body, and binding ends 124,224 of binding straps 120,220 may extend distally away from tension anchors 122,222. The piece of lingerie may be one or more of a brassiere, a panties, a corset, a garter belt, a choker necklace, and so forth. The specific anatomical locations may be at one or more of along the spine of the wearer, along or below the sternum of the wearer and collinear therewith, and along the sides of the torso of the wearer, as described in further detail below.

The main body of the piece of lingerie may be made from any desired material such as, but not limited to, silk, satin (which may be made from genuine silk, cotton, polyester, or other synthetic materials), genuine or imitation leather, genuine or imitation suede, velvet, chiffon, tatting, or other suitable lingerie materials generally as known in the art. The stretch percentage of the main body may be in the range of 0% to 40%, in the range of 10% to 40%, or in the range of 0 to 9.9%. The higher range for the stretch percentage may present a typical lingerie feel to the main body of the lingerie, while the lower range for the stretch percentage can increase the feeling of binding for the wearer, and introduce an amount of breath constriction for the wearer, thereby increasing sensation.

The binding straps may be attached to the piece of lingerie in various manners. In some exemplary embodiments, the binding straps may be permanently attached to the main body of the piece of lingerie, for example by being sewn thereto. In other exemplary embodiments, the binding straps may be attached to the piece of lingerie by coupling elements 130 that are in turn removably or permanently attached to the main body of the piece of lingerie. Such coupling elements may include round or oval rings, fabric loops, square rings, triangular rings, D-rings, 8-rings, strap adjusters, hooks, and so forth as generally known to one skilled in the art. In some exemplary embodiments, the binding straps may cooperate with the coupling elements so as to modify the shape of the piece of lingerie, for example, loosening or tightening the piece of lingerie around the body of the wearer or adapting the dimensions of the piece of lingerie to the body of the wearer.

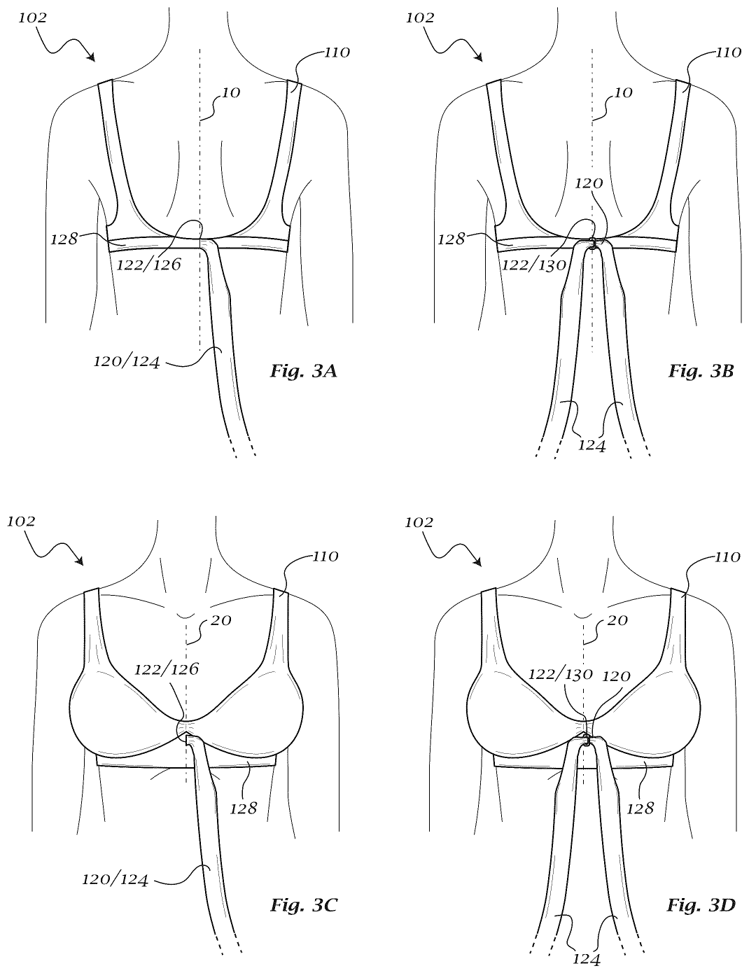

When the piece of lingerie is worn by a wearer, and a binding strap is attached to the piece of lingerie, each binding strap can include a tension anchor proximate to the body of the wearer, and at least one binding end extending distally away from the tension anchor. As used herein, a “tension anchor” is a location on the binding strap to which the tension from the binding end or binding ends of the binding strap is initially transmitted, or which provides a counteracting force to a pulling of the binding end or binding ends of the binding strap. For example, in the embodiments of FIGS. 3A, 3C, 4A, 4C, 4E, 4F, 5A, 5C, 5E, and 5F, where the binding strap 120 is permanently attached to the piece of lingerie 100, the tension anchor 122 is at the point of attachment 126 and the binding end 124 extends distally away therefrom. Similarly, in the embodiments of FIGS. 1, 3B, 3D, 4B, 4D, 4G, 4H, 5A, 5C, 5G, and 5H, where the binding strap 120 is attached to the piece of lingerie via a single coupling element 130, the tension anchor 122 is at the interface between the binding strap 120 and the coupling element 130 and the binding ends 124 extend distally away therefrom. A knot may also be formed at the single coupling element so as to maintain the binding strap fixed relative to the coupling element.

As another example, in the embodiments of FIGS. 2 and 6-8, the binding strap 220 is attached to the piece of lingerie via several coupling elements 230. In such embodiments, the binding strap may also be used to tighten or loosen the piece of the lingerie around the body of the wearer. That is, the distance between portions of the main body of the piece of lingerie may be increased or decreased. This may allow the piece of lingerie to be adapted to various body types, and may also allow the main body of the piece of lingerie to provide a constricting effect to the wearer when the piece of lingerie is tightened. Subsequently, a tension anchor 222 may be formed in the binding strap 220 substantially at the midpoint between the coupling elements 230 so as to maintain the desired tension on the piece of lingerie 200. The binding ends 224 of the binding straps 220 extend distally away from the tension anchor 222. In the depicted embodiments, the tension anchor 222 may be a knot. However, in other exemplary embodiments, the tension anchor may be formed utilizing other elements, such as a cord or lanyard lock, a cord slider, a clamp or clasp, a buckle, or any other such elements known in the art that allow the tension anchor to function as described herein. In all embodiments described herein, the binding end 124,224 of the binding strap 120,220, i.e., the portion of the binding strap 120,220 between the tension anchor 122,222 and the free distal end of the binding strap, has a length sufficient to bind at least one limb of the wearer to itself (i.e., said limb), or to another limb of the wearer. Furthermore, in such embodiments, the binding strap or straps may be removed and the coupling elements may be joined with a short strap or connector, allowing the piece of lingerie to be worn as a regular piece of lingerie.

According to the embodiments disclosed herein, each binding strap may be attached to the piece of lingerie such that the tension anchor of the binding strap is positioned proximate a specific anatomical location on the body of the wearer when the piece of lingerie is worn.

The binding straps may have a substantially elongated and planar configuration. The strap may be substantially consistently planar along its length and may have edges, for example finished edges that do not present any sharp or abrasive feel, thereby increasing comfort for the wearer.

The binding straps may have a length of between 1.0 m and 3.0 m, for example 1.0-2.0 m, 2.0-3.0 m, 1.0 m, 1.5 m, 2.0 m, 2.5 m, and 3.0 m, or any intermediate lengths therebetween. Such a range of lengths of the binding straps provides for sufficient strap length to position the wearer in a variety of different bound positions while still keeping the length of the strap manageable and not excessive such that it interferes with the execution and/or the aesthetics of the bound positions. Additionally, in some exemplary embodiments, if it is desired to lengthen a binding strap, an additional binding strap may be coupled to a binding end thereof. The coupling may be facilitated by a knot, or by other known coupling elements such as buttons, loops, snaps, and so forth. The binding straps may have a range of width between 3.0 cm and 40 cm, for example 3.0-25 cm, 25-30 cm, 3.0 cm, 5.0 cm, 7.0 cm, 10 cm, 15 cm, 20 cm, 25 cm, 30, cm, 35 cm, and 40 cm, or any intermediate lengths therebetween. In some exemplary embodiments, the width of the binding straps may vary along their length; for example, the width may gradually increase towards the binding end or ends of the binding strap. The width of the binding straps, in combination with the planar configuration of the strap, allows to provide a comfortable and gentle body feel in a bound position without imparting excessive pressure to the skin, muscles, lymph nodes, and other internal tissues. This may allow the wearer to spend longer amounts of time in bound positions with less fatigue and less pain, further allowing for muscle relaxation in the bound positions. The stretch percentage of the binding straps may be in the range of 0% to 25%, or, more preferably, in the range of 0% to 7%. The lower range for the stretch percentage can increase the ease of binding, and further maintain the wearer bound, while still providing a small amount of give so as to reduce discomfort.

The binding straps may be made from any desired material such as, but not limited to, silk, satin (which may be made from genuine silk, cotton, polyester, or other synthetic materials), genuine or imitation leather, genuine or imitation suede, velvet, chiffon, or other suitable lingerie materials generally as known in the art.

In some exemplary embodiments, as shown in FIGS. 3-5, each binding strap may be permanently attached to the piece of lingerie or may be attached to the piece of lingerie via a single coupling element. In such embodiments, the tension anchor of each binding strap may be disposed at the attachment point or at the coupling element, respectively, either of which may be positioned such that the tension anchor is disposed at a location that corresponds to a specific anatomical location on the human body when the piece of lingerie is worn by a wearer. Such embodiments are discussed further in detail below.

In some exemplary embodiments, as shown in FIGS. 3A-3D, when the piece of lingerie is a brassiere 102, the tension anchor 122 of a binding strap 120 may be disposed at a laterally central rear location of the brassiere, such that it disposed over or substantially proximate the spine 10 of the wearer when the brassiere is worn. In further exemplary embodiments, when the piece of lingerie is a brassiere 102, the tension anchor 122 of a binding strap 120 may be disposed at a laterally central front location of the brassiere, such that it is disposed over or substantially proximate the sternum 20 of the wearer when the brassiere is worn. In the aforementioned exemplary embodiments of the brassiere, the tension anchor of the binding strap may be disposed in vertically central or lower locations of the brassiere, i.e., for example, substantially at the connection between the two cups of the brassiere, or substantially at the connection of the rear straps of the brassiere. Further exemplary embodiments of the brassiere may include a tension anchor in both locations described above. The main body of the brassiere may include a band 128, to which the binding strap or single coupling element may be coupled. The band 128 may be reinforced and/or elastic, and may be made from a different material than the main body of the brassiere. The band 128 may receive the tension from the tension anchor such that the tension is not spread to the entirety of the main body of the brassiere, which may be more delicate. The band 128 may also facilitate spreading tension from the tension anchor to the ribcage of the wearer rather than the breasts.

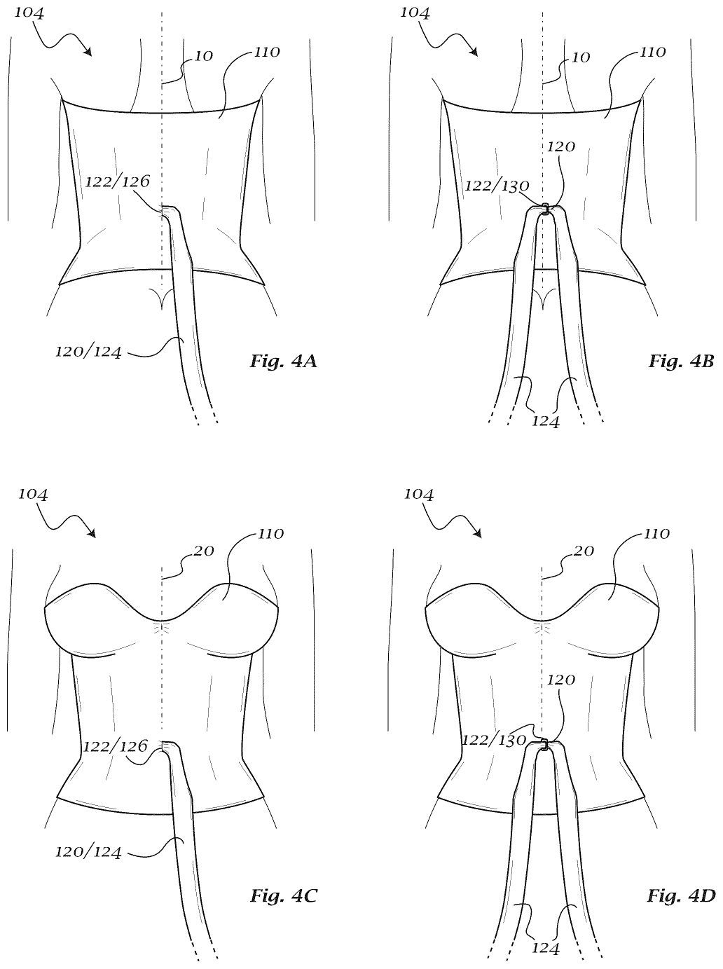

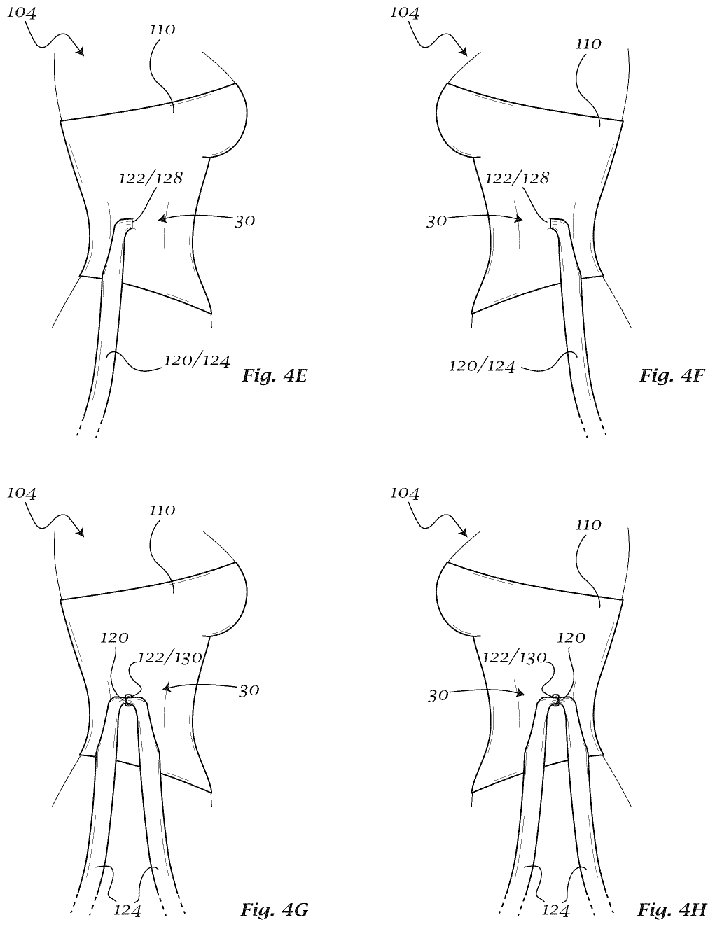

In some exemplary embodiments, as shown in FIGS. 4A-4H, when the piece of lingerie is a corset 104, the tension anchor 122 of a binding strap 120 may be disposed at a laterally central rear location of the corset, such that it disposed over or substantially proximate the spine 10 of the wearer when the corset is worn. In further exemplary embodiments, when the piece of lingerie is a corset 104, the tension anchor 122 of a binding strap 120 may be disposed at a laterally central front location of the corset, such that it is disposed over, substantially proximate the sternum 20, or below the sternum (epigastric or umbilical region), of the wearer when the corset is worn. In further exemplary embodiments, when the piece of lingerie is a corset 104, tension anchors 122 of two binding straps 120 may be disposed at opposing side locations of the corset, such that they are disposed over, or below, the left and right sides of the ribcage 30 of the wearer when the corset is worn. In the aforementioned exemplary embodiments of the corset, the tension anchor of the binding strap may be disposed, in various locations along the vertical direction of the corset, for example substantially at the bottom, substantially at the top, substantially at a vertically central location, or locations therebetween. Further exemplary embodiments of the corset may include tension anchors in two or more locations described above, or any combination thereof.

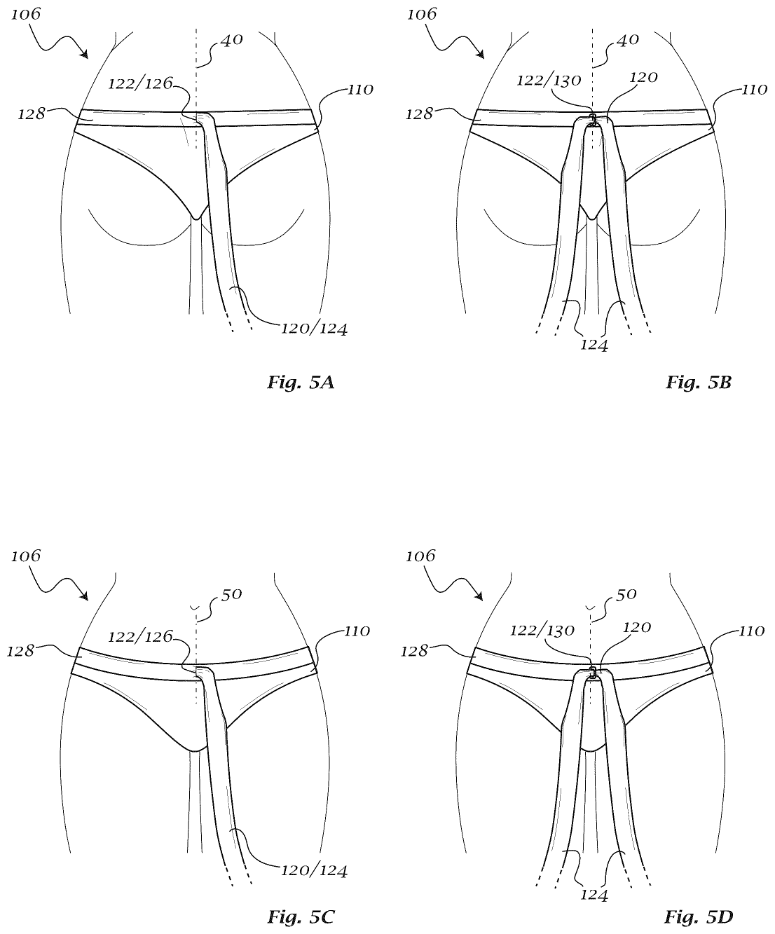

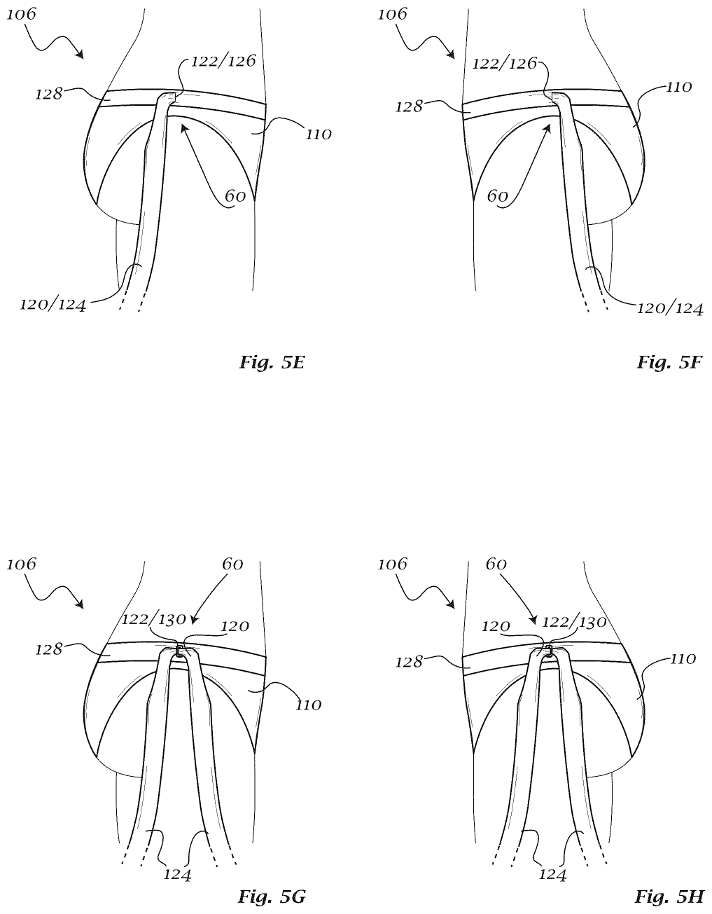

In some exemplary embodiments, as shown in FIGS. 5A-5H, when the piece of lingerie is a panties 106, the tension anchor 122 of a binding strap 120 may be disposed at a laterally central rear location of the panties, such that it disposed over or substantially proximate the lower spine 40, sacrum, or coccyx of the wearer when the panties are worn. In further exemplary embodiments, when the piece of lingerie is a panties 106, the tension anchor 122 of a binding strap 120 may be disposed at a laterally central front location of the panties, such that it is disposed below the navel and over or substantially proximate to the pubic (hypogastric) region 50 of the wearer when the panties are worn. In further exemplary embodiments, when the piece of lingerie is a panties 106, tension anchors 122 of binding straps 120 may be disposed at opposing side locations of the panties, such that they are disposed over the left and right sides of the iliac or lumbar regions 60 of the wearer when the panties are worn, for example over or substantially proximate the upper end of the ilium. Further exemplary embodiments of the panties may include attachment points in two or more locations described above, or any combination thereof. The main body of the panties may include a band 128, to which the binding strap or single coupling element may be coupled. The band 128 may be reinforced and/or elastic, and may be made from a different material than the main body of the brassiere. The band 128 may receive the tension from the tension anchor such that the tension is not spread to the entirety of the main body of the panties, which may be more delicate.

In some exemplary embodiments, when the piece of lingerie is a garter belt, the tension anchor of a binding strap or binding straps may be the same or substantially similar to the exemplary embodiments discussed above, when the piece of lingerie is a panties.

In some exemplary embodiments, when the piece of lingerie is a choker necklace, the tension anchor of a binding strap may be disposed at a laterally central rear location of the choker necklace, such that it disposed over or substantially proximate the neck vertebrae of the spine of the wearer when the choker necklace is worn.

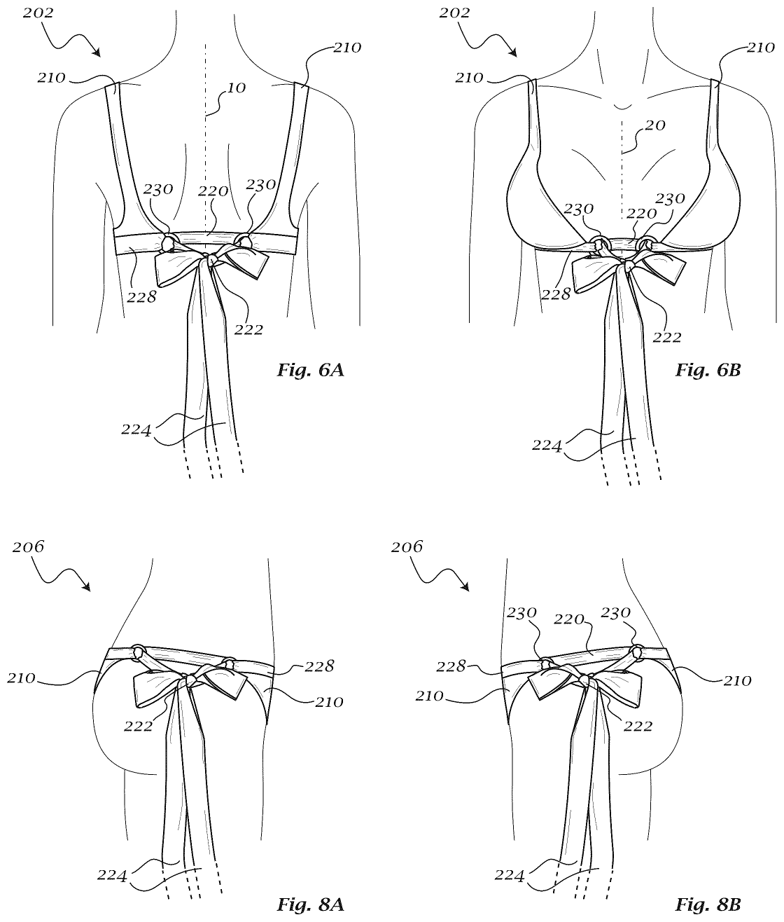

In yet further exemplary embodiments, as shown in FIGS. 6-8, the binding straps may further couple portions of the main body of the piece of lingerie. In such embodiments, the binding straps attach to the piece of lingerie via pairs of coupling elements. In such embodiments, the binding strap may also be used to tighten or loosen the piece of the lingerie around the body of the wearer, and so a tension anchor is formed in the binding strap substantially at the midpoint between the coupling elements so as to maintain the desired tension on the piece of lingerie. The binding ends of the binding straps extend distally away from the tension anchor. In the depicted embodiments, the tension anchor 222 may be a knot. However, in other exemplary embodiments, the tension anchor may be formed utilizing other elements, such as a cord or lanyard lock, a cord slider, a clamp or clasp, or any other such elements known in the art that allow the tension anchor to function as described herein. The tension anchor may receive tension from the binding ends of the binding strap when the binding straps are in use, and may provide a counteracting force to a pulling of the binding end or binding ends of the binding strap. Similarly, the tension anchor may receive tension from the coupling elements via the section of the binding strap is disposed between a coupling element and the tension anchor and may provide a counteracting force to a pulling by the coupling elements via the section of the binding strap that is disposed between a coupling element and the tension anchor.

In such exemplary embodiments, as shown in FIGS. 6A-6B, when the piece of lingerie is a brassiere 202, a binding strap 220 may couple the cups or rear straps of the brassiere 202 at the back of the wearer. The tension anchor 222 of binding strap 220 may be disposed at a laterally central rear location between the associated coupling elements 230 of the brassiere, such that it disposed over or substantially proximate the spine 10 of the wearer when the brassiere is worn. In further exemplary embodiments, when the piece of lingerie is a brassiere 202, a binding 220 strap may couple the cups of the brassiere at the front of the wearer. The tension anchor 222 of binding strap 220 may be disposed at a laterally central front location between the associated coupling elements 230 of the brassiere, such that it is disposed over or substantially proximate the sternum 20 of the wearer when the brassiere is worn. In the aforementioned exemplary embodiments of the brassiere, the tension anchor of the binding strap may be disposed, in vertically lower locations of the brassiere, i.e., for example, substantially between the two cups of the brassiere in the front, or substantially between the cups or the rear straps of the brassiere. Further exemplary embodiments of the brassiere may include tension anchors in both locations described above. The main body of the brassiere may include a band 228, to which the coupling elements may be coupled. The band 228 may be reinforced and/or elastic, and may be made from a different material than the main body of the brassiere. The band 228 may receive the tension from the tension anchor such that the tension is not spread to the entirety of the main body of the brassiere, which may be more delicate. The band 228 may also facilitate spreading tension from the tension anchor to the ribcage of the wearer rather than the breasts.

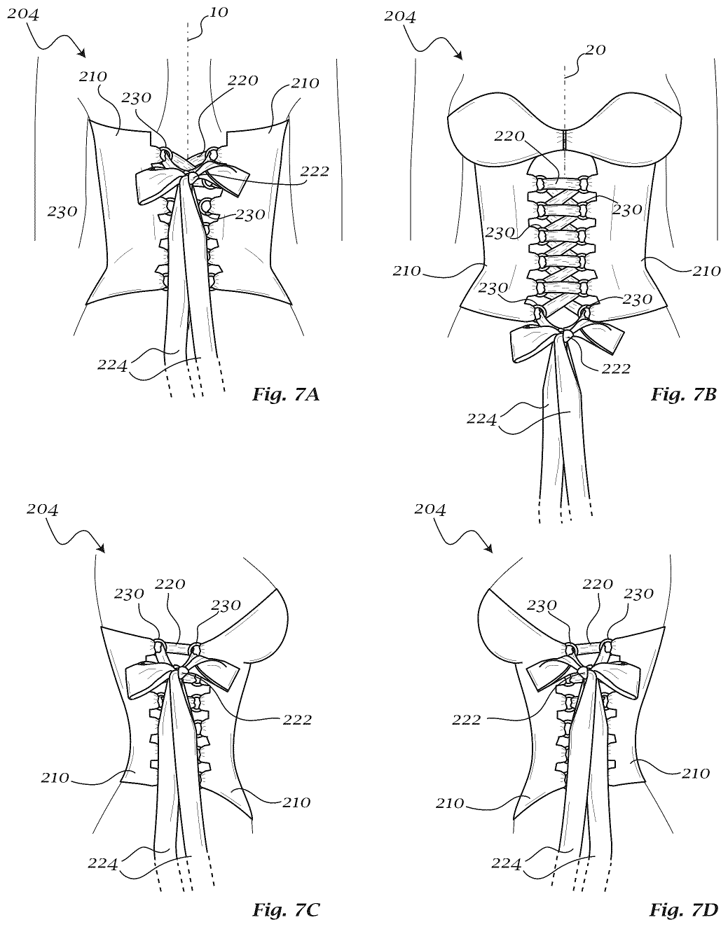

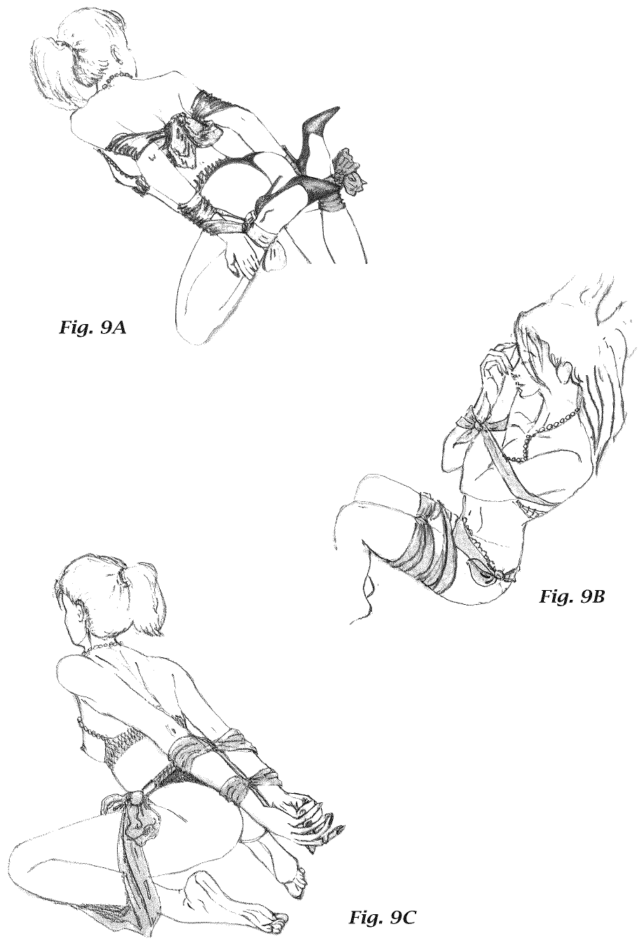

In some exemplary embodiments, as shown in FIGS. 7A-7C, when the piece of lingerie is a corset 204, a binding strap 220 may couple portions of the corset 204 at the back of the wearer. The tension anchor 222 of a binding strap 220 may be disposed at a laterally central rear location between the associated coupling elements 230 of the corset, such that it disposed over or substantially proximate the spine 10 of the wearer when the corset is worn. In further exemplary embodiments, when the piece of lingerie is a corset 204, a binding strap 220 may couple portions of the corset at the front of the wearer. The tension anchor 222 of a binding strap 220 may be disposed at a laterally central front location between the associated coupling elements 230 of the corset, such that it is disposed over, substantially proximate the sternum 20, or below the sternum (epigastric or umbilical region), of the wearer when the corset is worn. In further exemplary embodiments, when the piece of lingerie is a corset 204, binding straps may couple portions of the corset at the sides of the wearer. The tension anchors 222 of binding straps 220 may be disposed at opposing side locations between the associated coupling elements 230 of the corset, such that they are disposed over, or below, the left and right sides of the ribcage of the wearer when the corset is worn. In the aforementioned exemplary embodiments of the corset, the tension anchor of the binding strap may be disposed, in various locations along the vertical direction of the corset, for example substantially at the bottom, substantially at the top, substantially at a vertically central location, or locations therebetween. Further exemplary embodiments of the corset may include tension anchors in two or more locations described above, or any combination thereof. Furthermore, in some exemplary embodiments, multiple attachment points may be present along the vertical direction but in the same lateral location i.e., for example, a series of coupling elements extending vertically along the length of the corset at a laterally central rear location of the corset, such that they are disposed over or substantially proximate to and extend along the spine of the wearer when the corset is worn. Similar arrangements may be provided at the front or the sides of the corset as well.

In some exemplary embodiments, as shown in FIGS. 8A-8B, when the piece of lingerie is a panties 206, binding straps 220 may couple portions of the panties at the sides of the wearer. Tension anchors 222 of binding straps 220 may be disposed at opposing side locations between the associated coupling elements 230 of the panties, such that they are disposed over the left and right sides of the iliac or lumbar regions of the wearer when the panties are worn, for example over or substantially proximate the upper end of the ilium. The main body of the panties may include a band 228, to which the coupling elements may be coupled. The band 228 may be reinforced and/or elastic, and may be made from a different material than the main body of the brassiere. The band 228 may receive the tension from the tension anchor such that the tension is not spread to the entirety of the main body of the panties, which may be more delicate.

It should be appreciated that the lingerie need not be limited to the exact embodiments described above, and combinations of such embodiments may be contemplated and provided as desired. For example, in another exemplary embodiment of a panties, the binding straps may be fixedly connected to the main body of the lingerie substantially proximate the perineal region, and may extend rearward, upwards and laterally so as to pass through coupling elements coupled to the main body of the lingerie and located substantially in the iliac region, and then further pass rearward substantially horizontally to form a tension anchor substantially in the region of the coccyx, with the binding ends extending distally from the tension anchor. In yet another exemplary embodiment of a panties, the binding straps may be fixedly coupled to the main body of the lingerie substantially proximate the iliac region, and extend rearwardly so as to pass to a coupling element connected to the main body of the piece of lingerie substantially in the region of the coccyx, wherein the main body passes through the perineal region. A tension anchor may then be formed substantially in the region of the coccyx, with the binding ends extending distally from the tension anchor.

In some exemplary embodiments, when the piece of lingerie is a garter belt, binding straps may couple portions of the sides of the garter belt via coupling elements. The tension anchor of a binding strap or binding straps may be provided the same or substantially similar to the exemplary embodiments discussed above, when the piece of lingerie is a panties.

In some exemplary embodiments, when the piece of lingerie is a choker necklace, a binding strap may couple portions of the choker necklace at the back of the wearer. The tension anchor of a binding strap may be disposed at a laterally central rear location between associated coupling elements of the choker necklace, such that it disposed over or substantially proximate the neck vertebrae of the spine of the wearer when the choker necklace is worn.

According to another exemplary embodiment, a method for utilizing a piece of lingerie in an intimate bondage setting is disclosed. As a first step, a piece of lingerie may be provided, the piece of lingerie having a main body and at least one binding strap coupled or couplable to the main body. As a second step, a wearer may don the piece of lingerie. When the lingerie is donned, a tension anchor of the binding strap may be disposed at or proximate a specific anatomical location on the body of the wearer. The specific anatomical location may be one or more of along the spine of the wearer, along or below the sternum of the wearer and collinear therewith, and along the sides of the torso of the wearer, as described in further detail hereinabove. As a third step, at least one binding end of the binding strap may be extended distally from the tension anchor. As a fourth step, the binding end of the binding strap may be used to bind at least one limb of the wearer to itself (i.e., said limb) or to another limb of the wearer. The binding end of the binding strap may have a length sufficient to perform the binding of the limb or limbs.

Advantages provided by the embodiments and methods disclosed herein can include supporting a wearer of the lingerie that is bound by the binding straps in correct positions, stretching of certain muscle groups, and compression and fixation of parts of the body. In addition, utilizing the embodiments disclosed herein provides an interaction between the tension imparted to the limbs of the wearer that are bound by the binding straps and the tension transferred to the piece of lingerie that is worn by the wearer. Furthermore, the width of the binding straps provides more comfort to the wearer without the likelihood of imparting excessive pressure and compression to parts of the body such as nerves, lymph nodes, internal organs, and other tissues.

By utilizing the embodiments of the lingerie and the methods disclosed herein, the wearer of the lingerie may be bound in various diverse positions. Certain elements of the various positions are described below in a merely exemplary and non-limiting way, so as to demonstrate advantages of the present embodiments when utilized for binding. For example, as shown in FIG. 8A, when the piece of lingerie is a brassiere and in positions where both arms of the wearer are bound to each other behind the wearer utilizing at least one binding strap extending from a tension anchor located along the spine of the wearer, several physical and emotional effects may result. The biceps branchi may be stretched, allowing the elbow to relax. Additionally, the chest muscles may be stretched and the chest may be opened, allowing for a greater volume of oxygen to be inhaled, thereby increasing sensitivity. The deltoid muscle may further be relaxed. Additionally, the nerves in the solar plexus may be activated, thereby activating the sympathetic nervous system, increasing adrenaline, blood flow, and sensitivity. Furthermore, as shown in FIG. 8A, when the piece of lingerie is a panties, and in positions where an arm of the wearer is bound to a corresponding leg of the wearer utilizing at least one binding strap extending from a tension anchor located over a corresponding lumbar or iliac region of the wearer, several physical and emotional effects may result. The legs are bent, stretching the quadriceps femoris muscle. Blood flow to the pelvic region may be increased and relaxation of the pelvic region may be increased, also increasing arousal. In combination with the bound configuration of the arms in FIG. 8A, the hips are opened, stretching the rectus abdominus and iliopsoas muscles, further increasing blood flow to and relaxation of the pelvic region.

As a further example, as shown in FIG. 8B, when the piece of lingerie is a brassiere and in positions where both arms of the wearer are bound in front of the wearer utilizing at least one binding strap extending from a tension anchor located along the sternum or spine of the wearer, several physical and emotional effects may result. The elbow may be bent such that the lower arms approach the chest, imitating a fetal position, thereby relaxing the wearer and inducing a sense of comfort and safety, further increasing trust and intimacy. Furthermore, as shown in FIG. 8B, when the piece of lingerie is a panties, and when the legs of the wearer are bound to each other utilizing at least one binding strap extending from a tension anchor located over a lumbar or iliac region of the wearer, sensitivity and arousal may be increased.

As a further example, as shown in FIG. 8C, when the piece of lingerie is a panties and in positions where both legs of the wearer are bound to themselves utilizing two binding straps extending from a tension anchors located over the left and right sides of the iliac or lumbar regions of the wearer, several physical and emotional effects may result. The sacrum may relax, and the gluteus maximus and gluteus medius muscles may be stretched. The abdominal lymph nodes may be activated, as well as several abdominal muscles such as the pectineus, adductor brevis, adductor longus. This may activate the parasympathetic nervous system, thereby reducing stress, and inducing a feeling of balance and calmness. Furthermore, as shown in FIG. 8C, when the piece of lingerie is a brassiere and in positions where both arms of the wearer are bound to each other behind the wearer utilizing at least one binding strap extending from a tension anchor located along the spine of the wearer, several physical and emotional effects may result. The chest may be opened, and the abdominal muscles may be activated. This may activate the sympathetic nervous system, increasing adrenaline and arousal.

Many other binding positions and binding configurations may be contemplated and provided as desired, and may result various physical and emotional effects, depending on the location of the binding anchor, the limbs and portions thereof that are bound, the positions of the wearer and of the limbs of the wearer, and so forth. The length of the binding straps also allows users to vary the distance between the bound limbs of the wearer, depending on the flexibility and comfort level of the wearer.

The embodiments disclosed herein may allow intimate partners to partake in a form of bondage that allows an increase in relaxation and intimacy. Furthermore, aesthetic effects may be enhanced by including attractive lingerie in the intimate setting. Additionally, by combining the bondage elements and the lingerie, bodily sensations may be heightened, psychologically therapeutic effects may be provided, and intimacy between the partners may further be enhanced. Accordingly, the embodiments disclosed herein provide a novel lingerie and novel methods of utilizing bondage elements in an intimate setting.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A piece of lingerie, comprising:
a main body of the piece of lingerie; and
at least one binding strap fixedly or removably coupled to the main body;
wherein, when the lingerie is worn on a body of a wearer:
the binding strap comprises a tension anchor proximate to the body of the wearer and at least one free binding end extending distally from the tension anchor;
the binding end is connected to the main body of the piece of lingerie at or via the tension anchor;
the tension anchor is disposed at or proximate a specific anatomical location on the body of the wearer; and
the binding end of the binding strap has a length sufficient to bind at least one limb of the wearer to said limb or to another limb of the wearer.

2. The piece of lingerie of claim 1, wherein:
the binding strap is fixedly coupled to the piece of lingerie at an attachment point; and
the tension anchor is disposed at the attachment point.

3. The piece of lingerie of claim 1, wherein:
the binding strap is detachably coupled to the piece of lingerie via a single coupling element; and
the tension anchor is disposed at the coupling element.

4. The piece of lingerie of claim 1, wherein:
the binding strap is detachably coupled to the piece of lingerie via at least one pair of coupling elements; and
the tension anchor is substantially at the midpoint between coupling elements of the at least one pair of coupling elements.

5. The piece of lingerie of claim 1, wherein the tension anchor is disposed over or substantially proximate to one or more of:
the spine of the wearer;
the sternum of the wearer, or below the sternum and collinear therewith; and
the sides of the torso of the wearer.

6. The piece of lingerie of claim 1, wherein the piece of lingerie is a brassiere and the tension anchor is disposed at one or more of:
a laterally central rear location of the brassiere, such that the tension anchor disposed over or substantially proximate the spine of the wearer when the brassiere is worn; and
a laterally central front location of the brassiere, such that the tension anchor disposed over or substantially proximate the sternum of the wearer when the brassiere is worn.

7. The piece of lingerie of claim 1, wherein the piece of lingerie is a panties and the tension anchor is disposed at one or more of:
a laterally central rear location of the panties, such that the tension anchor disposed over or substantially proximate the lower spine, sacrum, or coccyx of the wearer when the panties are worn;
a laterally central front location of the panties, such that the tension anchor disposed over or substantially proximate the pubic region of the wearer when the panties are worn; and
opposing side locations of the panties, such that tension anchors are disposed over left and right sides of the iliac or lumbar regions of the wearer when the panties are worn.

8. The piece of lingerie of claim 1, wherein the binding strap has a length between 1.0 m and 3.0 m and a width between 3 cm and 40 cm.

9. The piece of lingerie of claim 1, wherein the binding strap has a stretch percentage in the range of 0% to 7%.

10. A piece of lingerie, comprising:
a main body of the piece of lingerie; and
at least one binding strap coupled or couplable to the main body;
wherein, when the lingerie is worn on a body of a wearer:
the binding strap comprises a tension anchor disposed at or proximate a specific anatomical location on the body of the wearer, and at least one free binding end extending distally from the tension anchor;
the tension anchor is coupled to the main body of the piece of lingerie at an attachment point or via at least one coupling element;
the binding end is connected to the piece of lingerie at or via the tension anchor;
the binding end of the binding strap has a length sufficient to bind at least one limb of the wearer to said limb or to another limb of the wearer; and
the specific anatomical location is one or more of along the spine of the wearer, along or below the sternum of the wearer and collinear therewith, and along the sides of the torso of the wearer.

11. The piece of lingerie of claim 10, wherein the piece of lingerie is a brassiere and the tension anchor is disposed at one or more of:
a laterally central rear location of the brassiere, such that the tension anchor disposed over or substantially proximate the spine of the wearer when the brassiere is worn; and
a laterally central front location of the brassiere, such that the tension anchor disposed over or substantially proximate the sternum of the wearer when the brassiere is worn.

12. The piece of lingerie of claim 10, wherein the piece of lingerie is a panties and the tension anchor is disposed at one or more of:
a laterally central rear location of the panties, such that the tension anchor disposed over or substantially proximate the lower spine, sacrum, or coccyx of the wearer when the panties are worn;
a laterally central front location of the panties, such that the tension anchor disposed over or substantially proximate the pubic region of the wearer when the panties are worn; and
opposing side locations of the panties, such that tension anchors are disposed over left and right sides of the iliac or lumbar regions of the wearer when the panties are worn.

13. The piece of lingerie of claim 10, wherein the binding strap has a length between 1.0 m and 3.0 m and a width between 3 cm and 40 cm.

14. The piece of lingerie of claim 10, wherein the binding strap has a stretch percentage in the range of 0% to 7%.

15. A method for utilizing a piece of lingerie in an intimate bondage setting, comprising:
providing a piece of lingerie having a main body and at least one binding strap coupled or couplable to the main body;
donning the piece of lingerie such that a tension anchor of the binding strap is disposed at or proximate a specific anatomical location on the body of the wearer;
extending at least one binding end of the binding strap distally from the tension anchor; and
binding, using the binding end of the binding strap, at least one limb of the wearer to said limb or to another limb of the wearer;
wherein the binding end of the binding strap has a length sufficient to perform the binding step; and
wherein the specific anatomical location is one or more of along the spine of the wearer, along or below the sternum of the wearer and collinear therewith, and along the sides of the torso of the wearer.

16. The method of claim 15, further comprising, after the donning step:
coupling the binding strap to the main body of the piece of lingerie via at least two coupling elements;
tightening or loosening the main body of the piece of lingerie to a desired tension using the binding strap;
forming the tension anchor so as to maintain the desired tension of the piece of lingerie; and
positioning the tension anchor substantially at the midpoint between the at least two coupling elements.

17. The method of claim 15, wherein the piece of lingerie is a brassiere and the tension anchor is disposed at one or more of:
a laterally central rear location of the brassiere, such that the tension anchor disposed over or substantially proximate the spine of the wearer when the brassiere is worn; and
a laterally central front location of the brassiere, such that the tension anchor disposed over or substantially proximate the sternum of the wearer when the brassiere is worn.

18. The method of claim 15, wherein the piece of lingerie is a panties and the tension anchor is disposed at one or more of:
a laterally central rear location of the panties, such that the tension anchor disposed over or substantially proximate the lower spine, sacrum, or coccyx of the wearer when the panties are worn;
a laterally central front location of the panties, such that the tension anchor disposed over or substantially proximate the pubic region of the wearer when the panties are worn; and
opposing side locations of the panties, such that tension anchors are disposed over left and right sides of the iliac or lumbar regions of the wearer when the panties are worn.

19. The method of claim 15, wherein the binding strap has a length between 1.0 m and 3.0 m and a width between 3 cm and 40 cm.

20. The method of claim 15, wherein the binding strap has a stretch percentage in the range of 0% to 7%.
